(19) United States
(12) Plant Patent Application Publication
(10) Pub. No.: US 2009/0113585 P1
(43) Pub. Date: Apr. 30, 2009

(54) Doritaenopsis plant named 'Moon N12'

(50) ;

(76) Inventor: Tsung-Te Huang, Xinying City (TW)

Correspondence Address: BIRCH STEWART KOLASCH & BIRCH, PO BOX 747, FALLS CHURCH, VA, 22040-0747, US

(21) Appl. No.: 11/979,057

(22) Filed: Oct. 30, 2007

(51) Int. Cl. A01H 5/00

(52) U.S. Cl. PLT/311

(57) ABSTRACT

A new and distinct cultivar of Doritaenopsis plant named ‘Moon N12’ characterized by its general impression of petals and sepals are white with light purple shadow near by the labellum flowers with purple light-colored labellum; upright and sturdy flowering stems, and excellent postproduction longevity.

### FIELD OF THE INVENTION

The present invention relates to botanical classification/cultivar designation: Doritaenopsis Orchid plant cultivar Moon N12.

### BACKGROUND OF THE INVENTION

The present invention comprises a new and distinct cultivar of Doritaenopsis Orchid, and hereinafter referred to by the cultivar name, ‘Moon N12’.

The new cultivar is a planned breeding program conducted by the Inventor in Tainan, Taiwan. The objective of the breeding program is to create new uniform pot-type Doritaenopsis Orchid cultivars having attractive flower coloration.

The new cultivar was discovered by the Inventor from within the progeny of a cross-pollination of two unidentified proprietary selections of Doritaenopsis and Phalaenopsis Orchids, not patented, on Oct. 18, 1999, in a controlled environment in Tainan, Taiwan.

Asexual propagation by tissue culture in a laboratory in Tainan, Taiwan has been used to increase the number of plants for evaluation and has demonstrated that the unique combination of characteristics as herein disclosed for the new Doritaenopsis Orchid are retained through successive generations of asexual reproduction.

### SUMMARY OF THE INVENTION

The following traits have been repeatedly observed and are determined to be basic characteristics of new cultivar which in combination distinguish this Doritaenopsis Orchid as a new and distinct cultivar:
1. Flower: Width in front view is broad about 11 to 13 cm.
2. Flowers persistent habit.
3. Upright, freely branching and sturdy flowering stems.
4. Excellent postproduction longevity.
Plants of the new cultivar differ primarily from plants of the parent cultivar in flower color.

Plants of the new Doritaenopsis Orchid can be compared to plants of the cultivar Maki Watanabe, disclosed in R.H.S. In side-side comparisons conducted in Tainan, Taiwan, plants of the new Doritaenopsis Orchid differed from plants of the cultivar Maki Watanabe in the following characteristics:
1. Plants of the new Doritaenopsis Orchid were bigger than plants of the cultivar Maki Watanabe.
2. Plants of the new Doritaenopsis Orchid had broader leaves than plants of the cultivar Maki Watanabe.
3. Plants of the new Doritaenopsis Orchid had shorter inflorescences than plants of the cultivar Maki Watanabe.
4. Plants of the new Doritaenopsis Orchid had strong peduncles than plants of the cultivar Maki Watanabe.

### BRIEF DESCRIPTION FOR THE DRAWINGS

Colors in the photographs may appear different from the color values that appear in the detailed botanical description which more accurately describe the new cultivar.

FIG. 1 is a side view of a plant of ‘Moon N12’ flowering in the pot of 12 cm.

FIG. 2 is a close-up view showing the characteristics of the flower.

FIG. 3 is a close-up view showing the characteristics of the leaf.

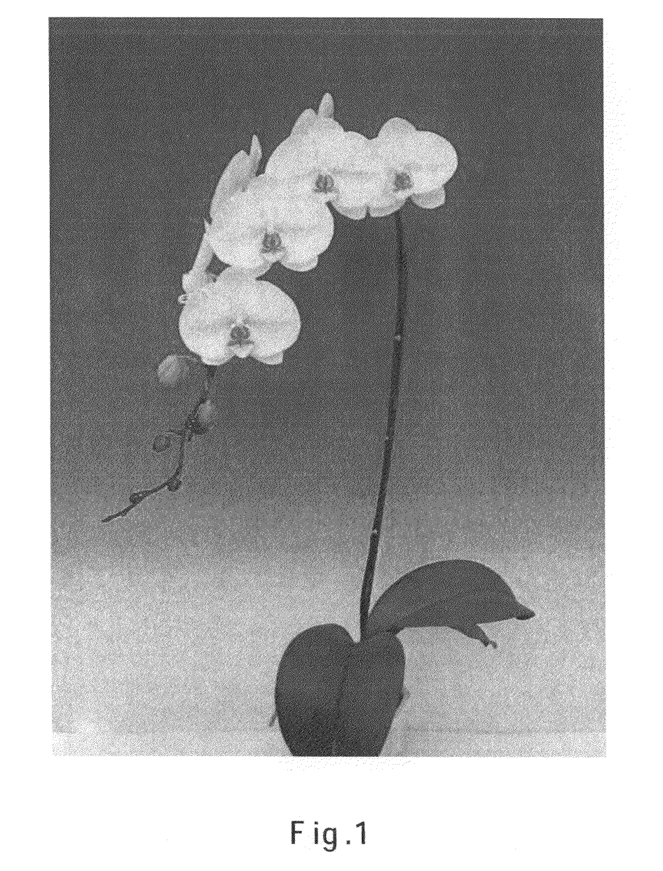

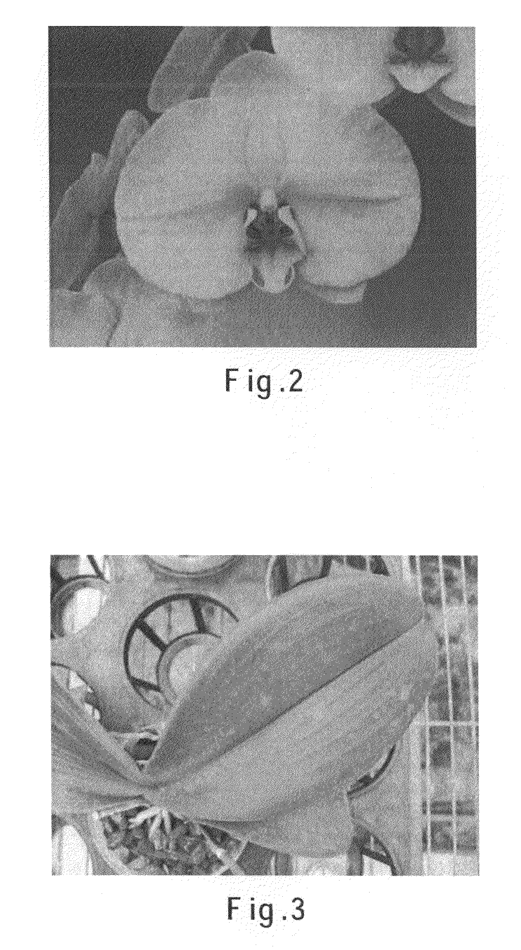

### DETAINED DESCRIPTION OF THE PREFERRED EMBODIMENTS

Parentage—Seed: Unidentified proprietary selection of Doritaenopsis, not patented.
Pollen: Unidentified proprietary selection of Phalaenopsis, not patented.
Propagation:
   Type.—Asexual propagation by tissue culture.
   Time to initiate and elongate roots.—Summer and winter: About 50 days at 27° C.
   Time to prodce a fully rooted young plant.—Summer: About 176 days at 27° C. Winter: About 192 days at 27° C.
Plant description:
   Form.—Two-ranked leaves affixed to a short central stem (monopodial growth). Single flowers arranged on upright and sturdy flowering compound racemes.
   Plant height, soil level to top of foliar plane.—About 15 to 20 cm.
   Plant height, soil level to top of inflorescences.—About 55 to 60 cm.
   Plant diameter.—About 35 to 40 cm.
   Flowers per stem.—Approximately 50 to 55.
Foliage description:
   Quantity per plant.—About 5 to 6.
   Length.—About 23 to 24 cm.
   Width.—About 9 to 10 cm.
   Shape.—Obovate.
   Apex.—Obtuse and rounded to retuse.
   Base.—Cuneate.
   Margin.—Entire.
   Aspect.—Mostly flat and folded upward form the midrib.
   Texture, upper and lower surfaces.—Leathery, thick, glabrous.
   Venation.—Parallel; veins are sunken within the lamina.
   Color.—Upper surface — Closest to 137A Lower surface — Closest to 146B.
Flower description:
   Flower type and habit.—Single zygomorphic flowers arranged in racemes; flowers roughly elliptical in shape. Flowering stems, upright. Plants typically produce one to two branched flowering stems with at least 10 to 11 flowers each. Flowers are not fragrant. Flowers persistent.
   Natural flowering season.—From January to May in southern Taiwan.
   Post-production longevity.—Plants of ‘Moon N12’ maintain good leaf and flower substance for about four to five months on the plant under interior environmental conditions.
   Inflorescence length.—About 42 to 45 cm.
   Inflorescence diameter.—About 38 to 40 cm.
   Flower diameter.—About 11 to 11.5 cm.
   Flower depth.—About 9 cm.
Petals:
   Quantity.—Two per flower.
   Length.—About 7.5 cm.
   Diameter.—About 5.5 cm.
   Shape.—Broadly ovate.
   Apex.—Rounded.
   Base.—Chunky; fused with the column.
   Margin.—Entire.
   Texture, upper and lower surfaces.—Velvety; smooth.
   Color.—Petal main color — 76D Petal pattern color — 84B.
Labellum:
   Length.—About 2 cm.
   Diameter.—About 2.3 cm.
   Shape.—Deeply three-lobed with two prominent callosities on the upper surface at the central junction of the lateral lobed and base of midlobe.
   Color.—Apical lobe base color — 85A Apical lobe tip color — N155B.
Sepals:
   Quantity.—Three per flower.
   Length.—About 5.5 cm.
   Diameter.—About 4.3 cm.
   Shape.—Elliptic to ovate.
   Apex.—Rounded.
   Base.—Attenuate; fused with the petals and column.
   Margin.—Entire.
   Texture, upper and lower surfaces.—Velvety; smooth.
   Color.—Dorsal sepal main color — 76B Dorsal sepal pattern color — 76B Lateral sepal main color — 76B Lateral sepal pattern color — 76B.
Peduncles:
   Length.—About 50 to 55 cm.
   Diameter.—About 5 mm.
   Aspect.—Upright.
   Strength.—Strong, sturdy.
   Texture.—Smooth, glabrous.
   Color.—200D.
Pedicels:
   Length.—About 4 cm.
   Diameter.—About 9 mm.
   Aspect.—About 75° from vertical.
   Strength.—Strong.
   Texture.—Smooth, glabrous.
   Color.—137A.
   Color towards the base.—137A.
Reproductive organs: Stamens and pistils into a column. Anthers with pollen grains united into pollinia. Stigma situated under the column and behind the pollinia.
Column:
   Length.—About 1.2 cm.
   Diameter.—About 5 mm.
   Color.—75D.
   Color towards to apex.—75D.
Pollinia:
   Quantity of pollen masses.—Two.
   Diameter.—About 1 mm.
   Color.—N25A.
Ovary:
   Length.—About 8 mm.
   Diameter.—About 2.5 mm.
   Color.—76D.
Root:
   Diameter.—About 4.5 mm.
   Color.—144A.
Plant disease resistance/susceptibility: Resistance to known pathogens and pests common to Doritaenopsis Orchids has not been observed on plants of the new cultivar grown under commercial greenhouse conditions.
Temperature tolerance: Plants of the new Doritaenopsis Orchid have been observed to be tolerant to temperatures from about 15 to 32□.

What is claimed is:

1. A new and distinct cultivar of Doritaenopsis plant named ‘Moon N12’, as illustrated and described.
